(19) United States
(12) Patent Application Publication
(10) Pub. No.: US 2006/0047254 A1
(43) Pub. Date: Mar. 2, 2006

(54) Phacoemulsification needle

(75) Inventor: Takayuki Akahoshi, Tokyo (JP)

Correspondence Address: ROTHWELL, FIGG, ERNST & MANBECK, P.C., 1425 K STREET, N.W., SUITE 800, WASHINGTON, DC, 20005, US

(73) Assignees: Ravi Nallakrishnan, Westmont, IL (US); Takayuki Akahoshi, Tokyo (JP)

(21) Appl. No.: 11/211,072

(22) Filed: Aug. 25, 2005

Related U.S. Application Data
(63) Continuation-in-part of application No. 10/866,187, filed on Jun. 14, 2004.
Application No. 10/866,187 is a continuation-in-part of application No. 10/406,571, filed on Apr. 4, 2003.

(30) Foreign Application Priority Data
Jun. 13, 2005 (EP) 05405378.0
Jun. 13, 2005 (JP) 2005-172884

(51) Int. Cl. A61M 5/32

(52) U.S. Cl. 604/272

(57) ABSTRACT

A phacoemulsification needle according to the invention comprises a shaft, a tip disposed at a distal end of said shaft and an aspiration lumen extending through said shaft and said tip. The tip has an opening communicating with said aspiration lumen. The shaft comprises at least a portion which has a ribbed outer surface. This needle minimizes heat generation and can therefore be used with or without sleeve.

### CROSS REFERENCE TO RELATED APPLICATIONS

This application is a Continuation-In-Part of U.S. application Ser. No. 10/866,187 filed on Jun. 14, 2004, which is a Continuation-In-Part of U.S. application Ser. No. 10/406,571 filed on Apr. 4, 2003.

### FIELD OF THE INVENTION

This invention relates to a phacoemulsification needle for an ultrasonic surgical instrument, the needle being designed for promoting cavitation in eye tissue and for the removal of fragmented lens from the eye.

### BACKGROUND OF THE INVENTION

Phacoemulsification has become the preferred form of cataract, i.e. a cloudy eye lens, removal. One of the main advantages of phacoemulsification is, that only a small incision into the cornea or sclera of an eye is needed to remove the cataract. Furthermore, the removal of the cataract can be done very quickly. After the cataract is removed, an intraocular lens is inserted to replace the original lens.

The phacoemulsification technique uses a hand held microsurgical tool known as phacoemulsifier. This phacoemulsifier comprises a handpiece and a small diameter needle with a tip to be inserted into the small incision of the eye. The needle and therefore the tip are vibrated by an ultrasonic source. It breaks the cataract into small fragments and pieces, which are sucked out through the same tip in a controlled manner. The tip is therefore designed for emulsifying, fragmenting and/or cutting tissue and also comprises a central hollow bore or lumen for the suction or aspiration of the fragments.

During the procedure, an irrigation solution is introduced to maintain the pressure and to prevent the eye from collapsing. In order to introduce the irrigation solution, the needle is usually covered by a sleeve and the solution flows via the space between this sleeve and the needle. The solution is therefore also used to cool the tip, which is heating up during the phacoemulsification.

Heat generated by the needle or tip degenerates the collagen of the cornea or sclera in the wound. If the collagen is degenerated by the heat, the wound will be damaged and cannot be self-sealed at the end of the surgery. An unsealed wound can cause anterior chamber collapse, which will result in severe corneal endothelial cell damage and most serious intraocular infection. Suturing the heat damaged tissue may attain sealing of the wound, however, it will cause deformity of the cornea and result in the large post-operative astigmatism. Thus -decreasing the risk of the thermal damage of the wound by the tip is one of the most important points of cataract surgery by phacoemulsification.

U.S. Pat. No. 5,653,724 discloses a phacoemulsification needle which is angled to provide a more comfortable ergonomic angle during phacoelmulsification and lens cortex removal. This angled needle is also considered to produce less heat when emulsifying the lens. Another angled phacoemulsification needle with a concentric sleeve is disclosed in U.S. Pat. No. 5,993,409.

Different shapes of phacoemulsification needles with slits, a second infusion hole and/or with increased outside diameter at the distal end of the needle body and the needles being surrounded by sleeves are described in U.S. Pat. Nos. 5,989,209, 6,159,175, EP-A-1,103,238, WO 00/74615 and US 2002/0099325. U.S. Pat. No. 5,989,209 furthermore discloses the use of a ribbed insert together with a sleeve. This shall permit continued irrigation fluid flow while reducing the contact area between the sleeve and the ribbed insert, thereby reducing the risk of thermal damage to the entry wound of the eye.

Other techniques for cataract removal use laser energy to remove the cataract. A laser/aspiration probe is used for breaking and removing the lens. A separate infusion or irrigation probe is used for the irrigation solution.

### SUMMARY OF THE INVENTION

It is an object of the invention to provide a phacoemulsification needle which minimizes the heat generation and which can therefore also be used without a sleeve.

According to a first aspect of the present invention, this object is achieved with a phacoemulsification needle comprising
a shaft,
a tip, disposed at a distal end of said shaft and
an aspiration lumen extending through said shaft and said tip, the tip having an opening communicating with said aspiration lumen,
wherein said shaft comprises at least one portion having a ribbed outer surface formed by spiral ridges or a helix.

According to a second aspect of the present invention, this object is achieved with a phacoemulsification needle comprising
a shaft with a first and a second end,
a tip, disposed at the first end of said shaft and
an aspiration lumen extending through said shaft and said tip, the tip having an opening communicating with said aspiration lumen,
wherein said shaft comprises at least one portion having protrusions or depressions in the shape of rings on its outer surface.

According to a third aspect of the present invention, this object is achieved with a phacoemulsification needle comprising
a shaft with a first end and a second end,
a tip, disposed at the first end of said shaft and
an aspiration lumen extending through said shaft and said tip, the tip having an opening communicating with said aspiration lumen,
wherein said shaft comprises at least one portion having protrusions on its outer surface, wherein each protrusion has a convex shape.

According to a fourth aspect of the present invention, this object is achieved with a phacoemulsification needle comprising
a shaft with a first end and a second end,
a tip, disposed at the first end of said shaft and
an aspiration lumen extending through said shaft and said tip, the tip having an opening communicating with said aspiration lumen,
wherein said shaft comprises at least one portion having depressions in its outer surface, wherein each depression has a concave shape.

According to a fifth aspect of the present invention, this object is achieved with a phacoemulsification needle comprising
a shaft with a first end and a second end,
a tip, disposed at the first end of said shaft and
an aspiration lumen extending through said shaft and said tip, the tip having an opening communicating with said aspiration lumen,
wherein said shaft comprises at least one grooved portion having a plurality of grooves in its outer surface, wherein said grooves have a helical form with a pitch angle between 3 and 30 degrees.

Since the shaft comprises depressions or protrusions, such as ribs or grooves, the surface of the tip is increased and the cooling effect of the tip is markedly enhanced.

In a preferred embodiment, the ribs or grooves have a spiral or helical form, or they are rings, or they are rectilinear or they have a concave or convex shape.

The needle according to the invention can be used with or without an irrigation sleeve.

In a preferred embodiment, the tip comprises a slit extending in the longitudinal direction of the needle and communicating with the aspiration lumen. When a high vacuum setting is used with the phacoemulsifier, the anterior chamber becomes unstable when occlusion break occurs. In this case, a small amount of irrigation fluid will continue to flow through the aspiration lumen. Furthermore, when occlusion surge occurs, the amount of surge can be reduced.

Further preferred embodiments of the invention are described in the dependent claims.

### BRIEF DESCRIPTION OF THE DRAWINGS

The present invention will be more clearly understood with reference to the following detailed description of preferred embodiments, taken in conjunction with the accompanying drawings, in which

FIG. 1a shows a perspective view of a needle according to the invention in a first embodiment,

FIG. 1b the needle according to figure la in a side view,

FIG. 1c in another side view,

FIG. 1d shows a magnified part of the needle tip according to FIG. 1c;

FIG. 2a shows a perspective view of a needle according to the invention in a second embodiment,

FIG. 2b the needle according to FIG. 2a in a side view,

FIG. 2c in another side view,

FIG. 2d shows a magnified front view of the needle according to FIG. 2a;

FIG. 2e shows a front view of the needle according to FIG. 2d;

FIG. 3a shows a perspective view of a needle according to the invention in a third embodiment,

FIG. 3b the needle according to FIG. 3a in a side view,

FIG. 3c in another side view,

FIG. 3d shows a magnified front view of the needle according to FIG. 3a and

FIG. 4a shows a perspective view of a needle according to the invention in a fourth embodiment,

FIG. 4b the needle according to FIG. 4a in a side view,

FIG. 4c in another side view,

FIG. 4d shows a magnified front view of the needle according to FIG. 4a;

FIG. 5 shows a perspective view of a needle according to the invention in a fifth embodiment;

FIG. 6 shows a perspective view of a needle according to the invention in a sixth embodiment;

FIG. 7 shows a perspective view of a needle according to the invention in a seventh embodiment;

FIG. 8 shows a perspective view of a needle according to the invention in an eighth embodiment;

FIG. 9a shows a perspective view of a needle according to the invention in a ninth embodiment; and

FIG. 9b shows side elevational view of the needle of FIG. 9a.

### DESCRIPTION OF THE PREFERRED EMBODIMENTS

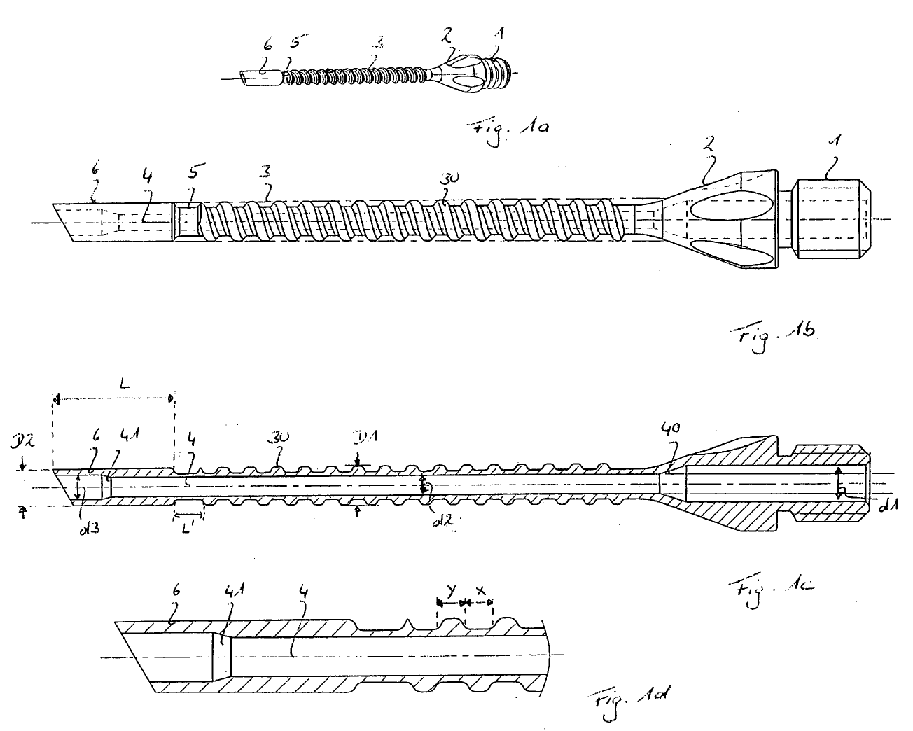

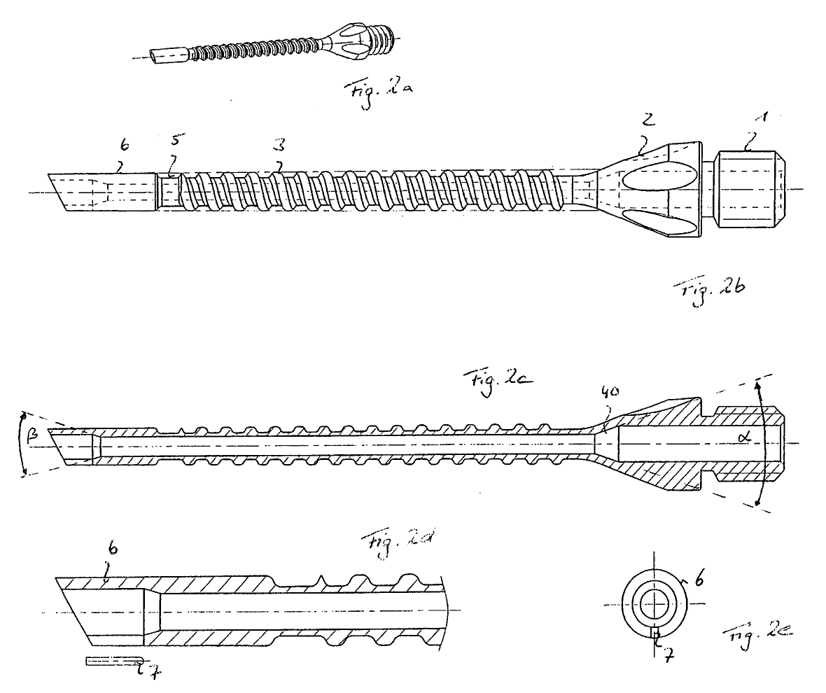

With reference to FIGS. 1a and 1b, there is shown a phacoemulsification needle in accordance with the present invention. The needle may be formed from any conventional material as is known in the art for the manufacture of phacoemulsification needles. Usually, it is made of titanium.

The needle comprises, starting with its proximal end and ending with its distal end (with reference to the surgeon), a threaded portion 1, a hub 2, a shaft 3 and a tip 6 having an opening. The threaded portion 1 and the hub 2 form a joint end enabling the needle to be fixed to a ultrasonic device of the phacoemulsification handpiece (not shown) in order to couple ultrasonic energy to the needle.

The needle further comprises, with reference to FIGS. 1c and 1d, an aspiration lumen 4 extending through the whole longitudinal length of the needle shaft 3 and communicating with the opening of the tip for aspiration of irrigation fluid and therefore for removal of fragments and pieces of the cataract. Preferably, the lumen 4 comprises a first step 40 in the region of the hub 2 neighboring the shaft 3. The first diameter d1 of the part of the lumen extending within the hub 2 is therefore larger than the second diameter d2 of the lumen part extending within the shaft 2. This first step 40 has preferably a conical shape.

A second step 41, which has preferably also a conical shape, is located within the tip 6, wherein the second diameter d2 is smaller than a third diameter d3 of the lumen part extending within the shaft 2. The width of the opening of the tip is even larger than the third diameter d3.

With exception of the steps 40, 41, the lumen preferably has throughout its whole length the same size. In particular the lumen part extending in the tip 6 has a cylindrical shape.

In this embodiment, the shaft 3 is rectilinear. The shaft 3 comprises at least one portion which has a ribbed outer surface 30. These ribs extending radially from the outer side of the shaft are preferably built by spiral ridges or a helix. Other protrusions are however possible. This portion extends preferably at least approximately along the whole length of the shaft 3.

Disposed between this portion with the ribbed surface 30 and the tip 6 is a tapered portion 5 (in other words, a constriction or a region of reduced diameter), connecting these two. In the embodiment shown, the tapered portion is immediately adjacent to the ribbed portion. The tip 6, which is disposed at the distal end of the shaft 3, has preferably at least approximately the same outer diameter D2 as the outer diameter D1 of the shaft 3. The above mentioned second step 41 is preferably located in the tip 6 itself at a distance to the transition of the ball-shaped surface 60 to the surface of the shaft 3.

A preferred embodiment of the inventive needle has an outer diameter D1 of the shaft 2 and an outer diameter D2 of the tip of approximately 1.32 mm, an inner diameter D1′ of the shaft 3 of approximately 0.956 mm, a first diameter d1 of the lumen 4 of approximately 1.32 mm, a second diameter d2 of approximately 0.72 mm, a third diameter d3 of approximately 0.9 mm, a length L of the tip 5 of approximately 4.385 mm and a distance x from the frontal end to the second step 41 of approximately 1.8 mm. The distance X between two ribs is approximately 0.5 mm, the thickness Y of a rib is also approximately 0.5 mm. The length L′ of the tapered portion 5 is approximately 0.8 mm, wherein the transition areas between the tapered portion and the tip and the tapered portion and the shaft respectively comprise an angle of 30° each. The angle a of the first step 40 is approximately 34° and the angle □ of the second step 41 approximately 30°. The total length of the needle is approximately 30 mm.

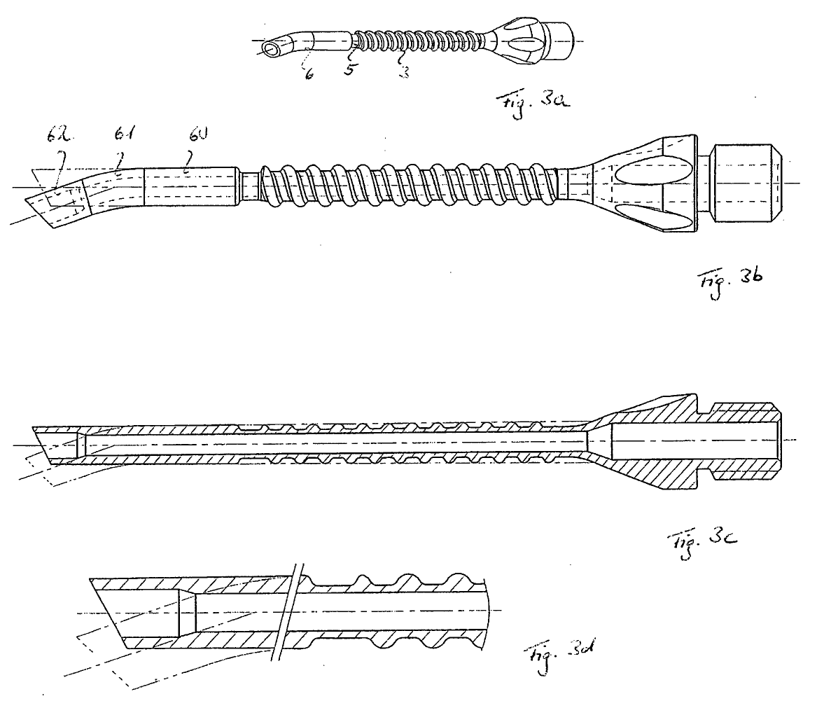

FIGS. 2a to 2d show a second preferred embodiment of the needle according to the invention. The needle is built in the same way as the needle according to the previously described figures. The only difference is, that this tip 6 comprises a slit 7 extending in longitudinal direction of the needle, which communicates with the aspiration lumen 4. This slit can be best seen in FIGS. 2d and 2e.

In both these embodiments, the ribbed surface has only a single ridge forming a single screw thread (in other words, a helix or a helical thread) along the ribbed surface. In other words, the ribbed surface may be described as a single-threaded screw surface. The pitch angle of the thread, i.e., the angle of the ridge relative to the long axis of the shaft, is relatively large. In particular, the ridge winds around the long axis of the shaft by a plurality of turns, in the present example approximately 15 turns. While a single-threaded surface is preferred, the surface may also be double-threaded, i.e., have two parallel ridges forming a double helix (in other words, two spiral ridges), or multiply-threaded.

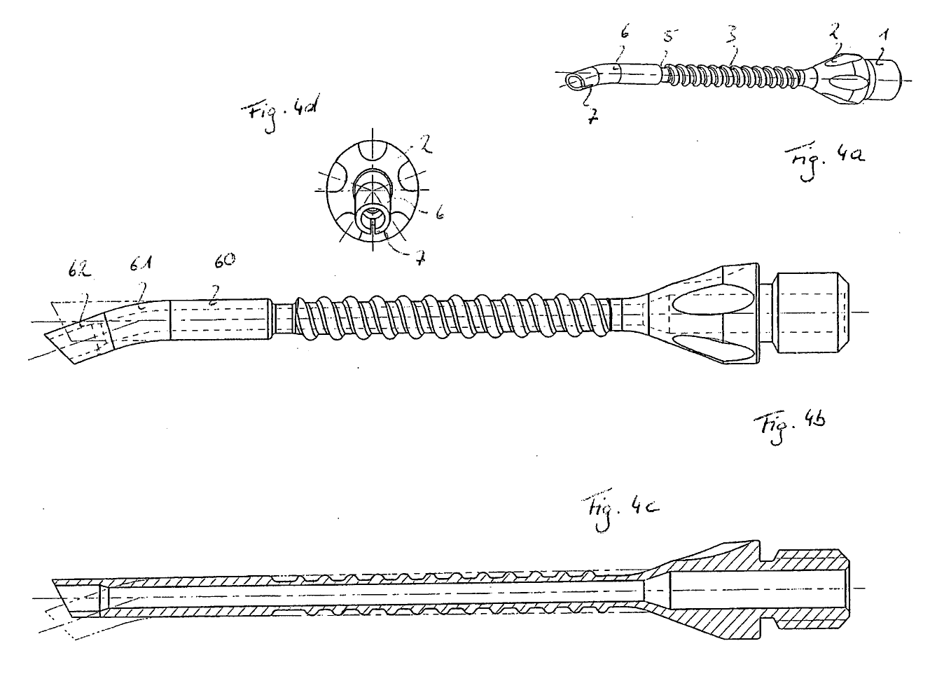

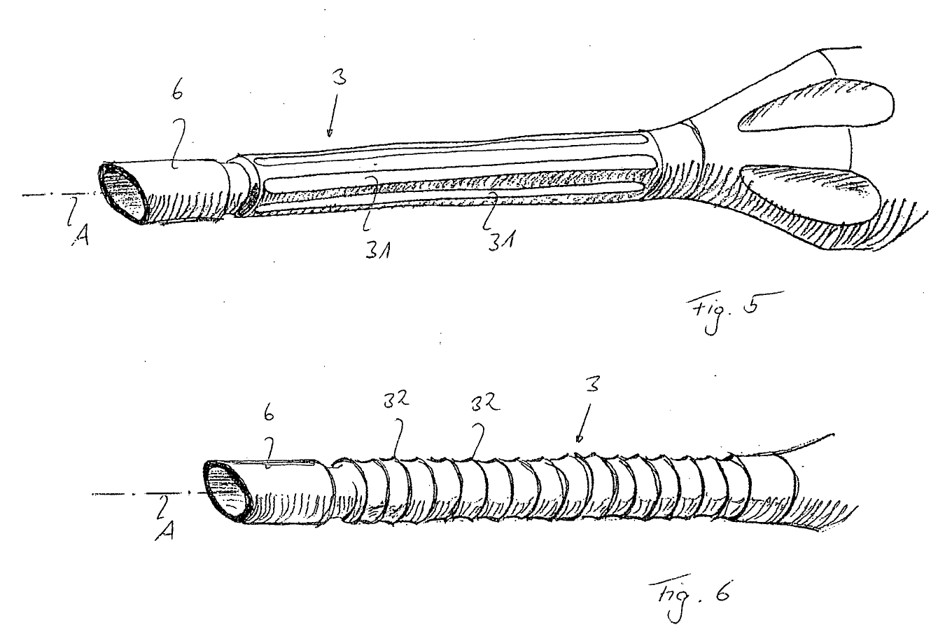

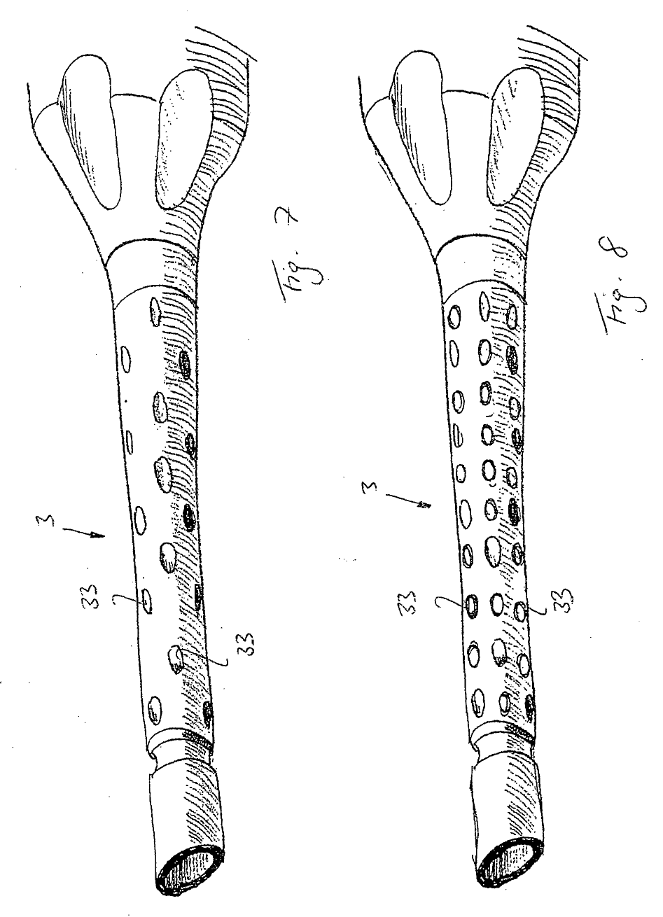

FIGS. 3a to 3d show a third preferred embodiment being similar to the embodiment according to FIGS. 1a to 1d without the slit 7 and FIGS. 4a to 4d show a fourth preferred embodiment being similar to the embodiment according to FIGS. 2a to 2d comprising the slit 7. The only difference to these figures is, that in the third and fourth embodiments the tip 6 is angled. The tip 6 consists of an extension portion 60 being disposed at the distal end of the tapered portion 5, followed by an angled portion 61 and an end portion 62. The end portion 62 has a flared end surface like the end portions of the first and second embodiments. This forms a large port or opening which can remove the lens material most efficiently, whereas the smaller diameter of the tip contributes to the stability of the anterior chamber formed by the lumen part within the tip. The angle between extension portion 60 and end portion 62 preferably lays between 17.5° and 20°.

FIG. 5 shows another preferred embodiment. In this embodiment, the shaft 3 comprises at least one portion which has one or more elongated grooves 31 extending at least approximately parallel to the longitudinal axis A of the needle shaft 3. In the embodiment shown the grooves 31 extend almost along the whole length of the shaft 3, they are rectilinear and they are arranged in an equidistant way from each other. However, these grooves 31 can also be arranged in a spiral (helical) way around the circumference of the needle shaft 3.

The embodiment according to FIG. 6 comprises ribs 32 or grooves, each of them having the shape of a ring or an annulus extending around the outer surface of the needle shaft 3. In the embodiment shown, the ribs 32 or grooves describe a plane being perpendicular to the longitudinal axis A of the shaft 3. However, they can also be inclined in an angle. Preferably, the ribs 32 or grooves are arranged in an equidistant way. However, the spaces between the ribs 32 or grooves can vary. For example, it is possible that the distances become smaller or bigger the nearer the ribs 32 or grooves are located to the distal end.

Further embodiments are shown in FIGS. 7 and 8. In these embodiments, the outer surface of the shaft 3 comprises a multiple of punctiform, concave depressions 33. These depressions 33 can all have the same size and depth or they can have different sizes and depths. Furthermore, it is possible that the sum of the depressed area amounts to almost half of the total surface area of the shaft or it can be far less than this amount.

The embodiments according to FIGS. 5 to 8 can comprise an angled tip 6 instead of the tip 6 extending along the longitudinal axis A.

Furthermore, the embodiments according to FIG. 7 and 8 can also comprise instead of or in addition to the concave depressions convex protrusions in the same arrangement and/or size as the depressions shown in these figures.

It is furthermore possible to combine depressions and protrusions, for example grooves and ribs, on the same shaft.

In another preferred embodiment, the ribs or grooves can also be located on the inner wall of the lumen 4. These inner ribs or grooves can be an additional feature of a tip having the outer ribs, helix or grooves. However, the tip can also comprise ribs and grooves on the inner side only and the outer surface can be smooth.

The needle according to the invention minimizes heat generation and can therefore be used with or without sleeve. In all of these designs the surface area of the needle is increased by the various designs to reduce heat with or without sleeve. When the needles are used in addition with a sleeve the irrigation may be enhanced due to the shape of the outer surface of the needle. When a sleeve extends over a smooth surface like the ones of conventional needles the flow of irrigation may be restricted which is not the case with the inventive needles. If a needle having a surface as shown in FIGS. 1 to 8 is used together with a sleeve irrigation will be increased because the space between the surface and the inner part of the sleeve is increased. However, especially the needles as shown in FIGS. 5, 7 and 8 are atraumatic to the tissue when they are used without a sleeve.

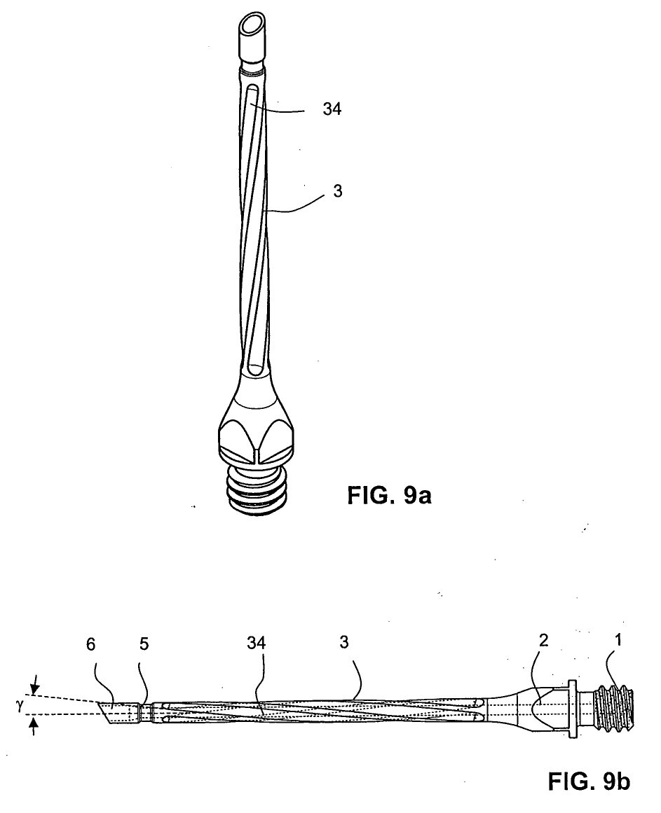

FIGS. 9a and 9b show a needle according to a yet another embodiment of the present invention. The shaft 3 of this needle comprises a portion with a grooved outer surface 30, in which a plurality of parallel and equidistant helical grooves is provided, the grooves having a small pitch angle. In particular, three, four or five parallel helical grooves are preferred. In the embodiment of FIGS. 9a and 9b, four grooves are provided.

It has been realized in the present invention that a reflux of anterior chamber fluid (aqueous humor) and/or externally supplied irrigation solution along the outer surface of the needle from the tip region of the needle towards the hub region may occur when the needle is used without a sleeve. This reflux has been be maximized in order to achieve optimum cooling of the outer surface for avoiding corneal or scleral damage. This was achieved by providing a plurality of ribs or grooves forming channels and being arranged in a helical shape, where the pitch angle (helix angle) is sufficiently small, but not zero. In particular, it has turned out that a pitch angle in the range of 3 to 30 degrees, preferably 5 to 15 degrees, most preferred 5 to 10 degrees, provides optimum reflux conditions. Grooves are preferred over ribs. Here, the pitch angle is defined as the angle of the ribs or grooves relative to the long axis of the shaft. In particular, FIGS. 9a and 9b show a needle in which helical grooves 34 are provided in the outer surface 30 of shaft 3 for which the pitch angle γ is approximately 8 degrees.

It is preferred that the pitch angle is constant along the length of the needle, i.e., that there is no variation in pitch angle. This provides optimum flow conditions for the reflux along the shaft.

The pitch angle, the diameter and the length of the needle are preferably chosen such that each groove winds around the long axis of the shaft by between approximately 0.3 and approximately 1.5 turns over the full length of the groove. More preferred is between approximately 0.5 turns and approximately one full turn. In the particular example of FIGS. 9a and 9b, the grooves make approximately 0.8 turns. This choice of the number of turns improves the reflux properties over the whole length of the grooved surface.

The grooves provided in the grooved outer surface 30 preferably have a rounded, in particular, semicircular, cross section. Their ends, viewed in an elevational view, are preferably blunted, in particular, rounded. The depth of the grooves preferably smoothly diminishes to zero towards the ends. In consequence of these measures, the cross section area of the grooves preferably diminishes smoothly to zero towards both ends. These measures all contribute to the formation of the reflux.

Also the needles according to the embodiment of FIGS. 9a and 9b preferably have a constriction 5 (a region of reduced diameter or, in other words, a tapered portion) arranged between the portion with the ribbed or grooved surface and the tip 6, connecting these two portions. This constriction is believed to aid in the formation of the reflux, improving the flow. Preferably, the constriction is slightly spaced from the ends of the grooves, where the distance between the ends of the grooves and the constriction is smaller than the diameter of the shaft.

The needles according to the present embodiment may have a slit for preventing pressure surges when occlusion break occurs, as described in connection with FIGS. 2a-2d and 4a-4d. Furthermore, the needles according to the present embodiment may have an angled tip, as described in connection with FIGS. 3a-3d and 4a-4d.

Due to its favorable properties, the needle according to the embodiment of FIGS. 9a and 9b is preferably used without a sleeve, even though it may also be used with a sleeve.

### LIST OF REFERENCE NUMBERS

1 threaded portion
2 hub
3 shaft
30 ribbed outer surface
31 grooves
32 ribs
33 depressions
34 helical grooves
4 lumen
40 first step
41 second step
5 tapered portion
6 tip
60 extension portion
61 angled portion
62 end portion
7 slit
A axis
d1 first diameter
d2 second diameter
d3 third diameter
D1 outside diameter of the shaft
D2 outside diameter of the tip
L length of the tip
L′ length of tapered portion
X distance between two ribs
Y thickness of a rib

1. A phacoemulsification needle comprising
a shaft,
a tip, disposed at a distal end of said shaft and
an aspiration lumen extending through said shaft and said tip, the tip having an opening communicating with said aspiration lumen,
wherein said shaft comprises at least one portion having a ribbed outer surface formed by spiral ridges or a helix.

2. The needle according to claim 1, wherein said helix is single-threaded.

3. The needle according to claim 1, wherein said helix winds around said shaft by a plurality of turns.

4. The needle according to claim 1, wherein said tip has an outer diameter being at least approximately the same as an outer diameter of said ribbed outer surface of said shaft.

5. The needle according to claim 1, wherein the needle comprises a tapered portion connecting said ribbed outer surface and said tip.

6. The needle according to claim 1, wherein said ribbed outer surface extends at least approximately along the whole length of said shaft.

7. The needle according to claim 1, wherein the tip is angled.

8. The needle according to claim 1, wherein said opening has a larger diameter than the part of the aspiration lumen extending within said shaft.

9. The needle according to claim 1, wherein said aspiration lumen comprises a conical step within the tip.

10. The needle according to claim 9, wherein said conical step has an angle of approximately 30°.

11. The needle according to claim 1, wherein said tip comprises a slit extending in longitudinal direction of the needle and communicating with said aspiration lumen.

12. A phacoemulsification needle comprising
a shaft with a first and a second end,
a tip, disposed at said first end of said shaft and
an aspiration lumen extending through said shaft and said tip, the tip having an opening communicating with said aspiration lumen,
wherein said shaft comprises at least one portion having protrusions or depressions in the shape of rings on its outer surface.

13. The needle according to claim 12, wherein the rings define an area being perpendicular to a longitudinal axis of the shaft.

14. The needle according to claim 12, wherein the rings are arranged equidistantly along a longitudinal axis of the shaft.

15. A phacoemulsification needle comprising
a shaft with a first and a second end,
a tip, disposed at said first end of said shaft and
an aspiration lumen extending through said shaft and said tip, the tip having an opening communicating with said aspiration lumen,
wherein said shaft comprises at least one portion having protrusions on its outer surface, wherein each protrusion has a convex shape.

16. A phacoemulsification needle comprising
a shaft with a first and a second end,
a tip, disposed at said first end of said shaft and
an aspiration lumen extending through said shaft and said tip, the tip having an opening communicating with said aspiration lumen,
wherein said shaft comprises at least one portion having depressions in its outer surface, wherein each depression has a concave shape.

17. A phacoemulsification needle comprising
a shaft with a first and a second end,
a tip, disposed at said first end of said shaft and
an aspiration lumen extending through said shaft and said tip, the tip having an opening communicating with said aspiration lumen,
wherein said shaft comprises at least one grooved portion having a plurality of grooves in its outer surface, wherein each said groove has a helical form with a pitch angle between 3 and 30 degrees.

18. The needle according to claim 17, wherein said grooves extend almost along the whole length of said shaft.

19. The needle according to claim 17, wherein said grooves are arranged in an equidistant way from each other.

20. The needle according to claim 17, wherein the number of grooves is selected from the numbers three, four and five.

21. The needle according to claim 17, wherein said pitch angle is constant along each said groove.

22. The needle according to claim 17, wherein each said groove winds around said shaft by between approximately 0.3 and approximately 1.5 turns.

23. The needle according to claim 17, wherein each said groove has a substantially rounded cross section.

24. The needle according to claim 17, wherein each said groove has substantially rounded ends.

25. The needle according to claim 17, wherein each groove has a first and a second end and wherein each groove has a cross-section area which smoothly diminishes towards said first and said second ends.

26. The needle according to claim 17, wherein said needle comprises a section having a reduced diameter between said shaft and said tip.

27. The needle according to claim 17, wherein said tip is angled.

28. The needle according to claim 17, wherein said tip comprises a slit extending in a longitudinal direction of the needle and communicating with said aspiration lumen.

29. A method of phacoemulsification comprising:
providing a needle having:
a shaft with a first and a second end;
a tip, disposed at said first end of said shaft; and
an aspiration lumen extending through said shaft and said tip, the tip having
an opening communicating with said aspiration lumen,
wherein said shaft comprises at least one portion having a ribbed outer surface formed by spiral ridges or a helix;
inserting said tip in an eye having a cataract;
vibrating said tip with substantially ultrasonic vibration;
breaking substantially said cataract into pieces with said tip; and
aspirating substantially said pieces through said aspiration lumen.

30. A method of phacoemulsification comprising:
providing a needle having:
a shaft with a first and a second end;
a tip, disposed at said first end of said shaft; and
an aspiration lumen extending through said shaft and said tip, the tip having
an opening communicating with said aspiration lumen,
wherein said shaft comprises at least one portion having protrusions or depressions in the shape of rings on its outer surface;
inserting said tip in an eye having a cataract;
vibrating said tip with substantially ultrasonic vibration;
breaking substantially said cataract into pieces with said tip; and
aspirating substantially said pieces through said aspiration lumen.

31. A method of phacoemulsification comprising:
providing a needle having:
a shaft with a first and a second end;
a tip, disposed at said first end of said shaft; and
an aspiration lumen extending through said shaft and said tip, the tip having
an opening communicating with said aspiration lumen,
wherein said shaft comprises at least one portion having protrusions on its outer surface, wherein each protrusion has a convex shape;
inserting said tip in an eye having a cataract;
vibrating said tip with substantially ultrasonic vibration;
breaking substantially said cataract into pieces with said tip; and
aspirating substantially said pieces through said aspiration lumen.

32. A method of phacoemulsification comprising:
providing a needle having:
a shaft with a first and a second end;
a tip, disposed at said first end of said shaft; and
an aspiration lumen extending through said shaft and said tip, the tip having
an opening communicating with said aspiration lumen,
wherein said shaft comprises at least one portion having depressions in its outer surface, wherein each depression has a concave shape;
inserting said tip in an eye having a cataract;
vibrating said tip with substantially ultrasonic vibration;
breaking substantially said cataract into pieces with said tip; and
aspirating substantially said pieces through said aspiration lumen.

33. A method of phacoemulsification comprising:
providing a needle having:
a shaft with a first and a second end;
a tip, disposed at said first end of said shaft; and
an aspiration lumen extending through said shaft and said tip, the tip having
an opening communicating with said aspiration lumen,
wherein said shaft comprises at least one grooved portion having a plurality of grooves in its outer surface, wherein said grooves have a helical form with a pitch angle between 3 and 30 degrees;
inserting said tip in an eye having a cataract;
vibrating said tip with substantially ultrasonic vibration;
breaking substantially said cataract into pieces with said tip; and
aspirating substantially said pieces through said aspiration lumen.
